(19) United States
(12) Patent Application Publication
(10) Pub. No.: US 2013/0144298 A1
(43) Pub. Date: Jun. 6, 2013

(54) BALLOON CATHETER

(71) Applicant: Gil Woon Choi, Wonju-si (KR)

(72) Inventor: Gil Woon Choi, Wonju-si (KR)

(21) Appl. No.: 13/327,062

(22) Filed: Dec. 15, 2011

(30) Foreign Application Priority Data
Dec. 5, 2011 (KR) 10-2011-0128741

(51) Int. Cl. A61B 17/56

(52) U.S. Cl. 606/93

(57) ABSTRACT

A balloon catheter is disclosed. The balloon catheter includes an outer tube, a solid rigid rod, a balloon, markers and a body unit. The rigid rod is inserted into the outer tube such that a gap is defined between the rigid rod and the inner surface of the outer tube. The front and rear ends of the rigid rod extend outwards from the outer tube. The front end of the balloon is fastened to the front end of the rigid rod. The rear end of the balloon is fastened to the front end of the outer tube. The markers are fastened to the rigid rod in the balloon at opposite positions spaced apart from each other. The body unit is fastened to the rear end of the outer tube and supports the rigid rod and prevents it from moving forwards.

### BACKGROUND OF THE INVENTION

1. Field of the Invention

The present invention relates to a balloon catheter which are used in the medical field and, more particularly, to a balloon catheter which can inflate the interior of a damaged portion, for example, of the spine of a patient, to allow medical cement for recovering the damaged portion to be injected into the damaged portion which is the result of a fracture, necrosis, a dent or the like.

2. Description of the Related Art

Generally, as a surgical procedure of treating, for example, the damaged spine of a patient, there has been a method of inserting a tubular catheter into a patient's body adjacent to the damaged spine of the patient and injecting medical cement into the damaged spine. Sufficiently inflating a damaged portion of the spine and injecting an appropriate amount of medical cement into the inflated portion are factors that are crucial to obtaining a satisfactory result in such surgical procedures using catheters for spine treatment.

FIG. 1 is a sectional view showing a typical balloon catheter. FIG. 2 is a partial sectional view showing the typical balloon catheter and an injector for spine surgical procedures which is connected to the balloon catheter.

As shown in FIG. 1, the balloon catheter 1 according to the conventional art includes a balloon 2 (or an inflation member), an outer tube 3, an inner tube 4, a guide wire 5, two markers 6 and a body unit 10. The body unit 10 includes a cap 11, a stylet 12, a body 13 and a holder 14.

The inner tube 4 is received inside the outer tube 3. A rear end of the outer tube 3 is fastened to and sealed to the cap 11. A rear end of the balloon 2 is fastened around and sealed to a front end of the outer tube 3. A front end of the balloon 2 is fastened around and sealed to a front end of the inner tube 4. An inside portion of the front end of the inner tube 4 is sealed by resin material. The two markers 6 are fastened to the inner tube 4 in the balloon 2. The two markers 6 function to indicate the location of the balloon 2 on the screen of a monitor when applying X-rays thereto, thus allowing a user to determine the location of the balloon 2.

The cap 11 is removably coupled to a front end of the body 13 and houses the stylet 12 therein. The stylet 12 enables the cap 11 to be reliably coupled to the body 13 and receives a corresponding portion of the inner tube 4. The body 13 receives therein a corresponding portion of the inner tube 4 and has at a predetermined position thereof an inclined port 13a which communicates with the outside. Further, the inner tube 4 is fastened to and sealed to an inner surface of a rear end of the body 13. The holder 14 is removably fastened to the rear end of the body 13. Moreover, a sealing ring 15 is interposed between the rear end of the body 13 and the holder 14 to keep fluid in the body 13 from leaking to the outside.

The guide wire 5 is fastened at a rear end thereof in a central portion of the holder 14 and inserted along the inner tube 4. The guide wire 5 functions to prevent the outer tube 3, to which the balloon 2 is attached, and the inner tube 4 in the outer tube 3 from possibly bending and impeding the surgical procedure, when the catheter 1 is inserted into a patient's body towards a damaged portion of the spine of the patient to perform the surgical procedure.

Furthermore, gaps are formed between the outer tube 3 and the inner tube 4, between the stylet 12 and the inner tube 4 and between the body 13 and the inner tube 4 so that high-pressure fluid can be supplied from the inclined port 13a of the body 13 into the balloon 2 through the gaps, thus inflating the balloon 2. The “high pressure of the fluid” means a pressure capable of inflating the balloon 2 to the appropriate size.

Meanwhile, the injector 20 which is shown in a lower portion of FIG. 2 includes a connection hose 21 which communicates with the outside, a cylinder 22 which is connected to the connection hose 21, and a plunger 23 which is inserted into the cylinder 22 with an elastic force applied thereto. The injector 20 further includes a grip 24 which is coupled to a rear end of the cylinder 22, a trigger 25 which allows the plunger 23 to move forwards, and a pressure meter 26 which indicates the pressure in the cylinder 22.

Next, a process of assembling the balloon catheter 1 having the above-mentioned construction with the injector 20 and the operation of the balloon catheter 1 will be explained with reference to FIG. 2.

First, after the plunger 23 of the injector 20 has been positioned at a rear end position of a cylinder 22, the connection hose 21 of the injector 20 is connected to the inclined port 13a of the catheter 1.

Subsequently, the catheter 1 is positioned in a target area of the spine of a patient through a catheter passage which has been previously formed in the patient's body by medical instruments. Here, because the guide wire 5 is disposed in the inner tube 4 of the catheter 1, the outer tube 3 and the inner tube 4 which are attached at the front ends thereof to the corresponding portion of the balloon 2 can be smoothly guided in the direction desired by a user rather than undesirably bending.

Thereafter, when the user pulls the trigger 25 of the injector 20, the plunger 23 is moved forwards, that is, towards the front end of the cylinder 22. Then, compressed air is supplied from the cylinder 22 to the inclined port 13a of the catheter 1 through the connection hose 21.

The compressed air that is supplied into the inclined port 13a of the catheter 1 flows along the gap between the outer tube 3 and the inner tube 4 via the gap between the stylet 12 and the inner tube 4 and enters the balloon 2 provided on the front end of the outer tube 3, thus inflating the balloon 2.

After the balloon 2 has been inflated by the above-mentioned process so that an available space is created in the target area of the spine of the patient, an X-ray examination is conducted, for example, to check whether a collapsed bone has returned to its normal location. Thereafter, the balloon 2 of the catheter 1 is extracted from the spine of the patient, and medical cement is injected into the target area of the spine that has recovered its normal form, thus completing the surgical procedure.

However, in the conventional balloon catheter 1 before a surgical procedure is performed, the guide wire 5 must be inserted into the inner tube 4 to prevent the outer tube 3, which the balloon 2 is attached at the front end thereof, and the inner tube 4 received in the outer tube 3, from bending while inserting the balloon catheter 1 into the patient's body.

Therefore, the conventional balloon catheter 1 is problematic in that it is very inconvenient for the user to have to insert the guide wire 5 into the inner tube 4 every time a surgical procedure is performed. In addition, the use of the inner tube 4 and the guide wire 5 makes the structure of the balloon catheter 1 complex.

Moreover, in the conventional balloon catheter 1 when high-pressure air is injected into the balloon 2 during the surgical procedure, the high pressure in the balloon 2 inflates the inner tube 4 and the balloon 2 in the longitudinal direction, resulting in irregularities in the radial inflation of the balloon 2. A reduction in the size of the radial inflation of the balloon 2 causes the problem of the size of the space into which medical cement can be inserted being reduced by more than is actually required by the space of the target area.

### SUMMARY OF THE INVENTION

Accordingly, the present invention has been made keeping in mind the above problems occurring in the prior art, and an object of the present invention is to provide a balloon catheter which has a simple structure and makes it possible to conveniently insert a balloon of the catheter into a target area of the spine of a patient without using an inner tube or a guide wire.

Another object of the present invention is to provide a balloon catheter which is configured such that high-pressure fluid, which is injected into the balloon to inflate it, is prevented from inflating a member for having a front end of the balloon attached or the balloon in the longitudinal direction, thus making it possible for the balloon to reliably inflate in the radial direction.

A further object of the present invention is to provide a balloon catheter which has an enhanced fixing strength between a front end of the balloon and a member to which the front end of the balloon is fastened and is configured such that fluid injected into the balloon to inflate it is structurally guided so that the pressure in the balloon is applied outwards in the radial direction.

In order to accomplish the above objects, in an embodiment, the present invention provides a balloon catheter, including: an outer tube; a rigid rod having a solid structure and inserted into the outer tube along a length of the outer tube such that a gap is defined between the rigid rod and an inner surface of the outer tube, the rigid rod having front and rear ends extending outwards from the outer tube by predetermined distances; a balloon fastened at a front end thereof around and sealed to the front end of the rigid rod, the balloon fastened at a rear end thereof around and sealed to a front end of the outer tube; markers fastened to the rigid rod in the balloon at opposite positions spaced apart from each other by a predetermined distance; and a body unit fastened to and sealed to a rear end of the outer tube and preventing the rigid rod from moving forwards, the body unit supporting the rear end of the rigid rod such that the gap is defined between the rigid rod and the outer tube so that fluid is supplied from an outside into the balloon through the gap between the outer tube and the rigid rod.

Furthermore, based on the above-described embodiment, the present invention may further provide the following embodiments.

In another embodiment, the rigid rod may be made of reinforced plastic or fiber-reinforced plastic.

In a further embodiment, the body unit may include: a cap fastened to and sealed to the rear end of the outer tube; a stylet housed in the cap and receiving the rigid rod therein such that a gap is defined between the stylet and an outer surface of the rigid rod; a body coupled both to a rear end of the cap and to a rear end of the stylet, the body supporting the rear end of the rigid rod, with an inclined port provided at a predetermined position on the body, the inclined portion communicating with the outside; and a holder coupled to a rear end of the body and sealed to the body by a sealing ring interposed therebetween, the holder retaining the rear end of the rigid body therein.

In yet another embodiment, the balloon catheter may further include a front tube provided between an inner surface of the front end of the balloon and the front end of the rigid rod to enhance a strength with which the balloon and the rigid rod are fixed to each other, the front tube comprising a pressure diffuser on a rear end thereof, the pressure diffuser including: a diffusion guide being increased in diameter from a front end thereof to a rear end; and a curved part formed on a central portion of the pressure diffuser, the curved part being convex outwards.

In still another embodiment, the rear end of the rigid rod that may be located in a space between the body and the holder has a stopper thereon or is fastened to the holder, thus preventing high-pressure fluid supplied into the balloon of the balloon catheter from moving the rigid rod forwards during a surgical procedure.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a sectional view showing a conventional balloon catheter;

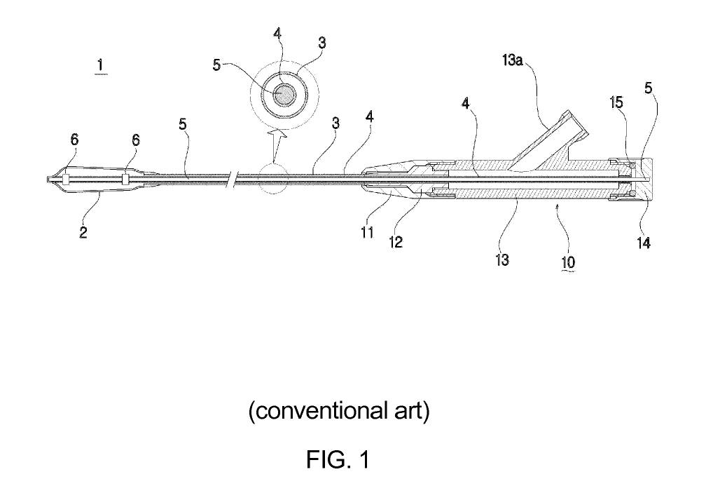

FIG. 2 is a partial sectional view showing the conventional balloon catheter and an injector for spine surgical procedures which is connected to the balloon catheter;

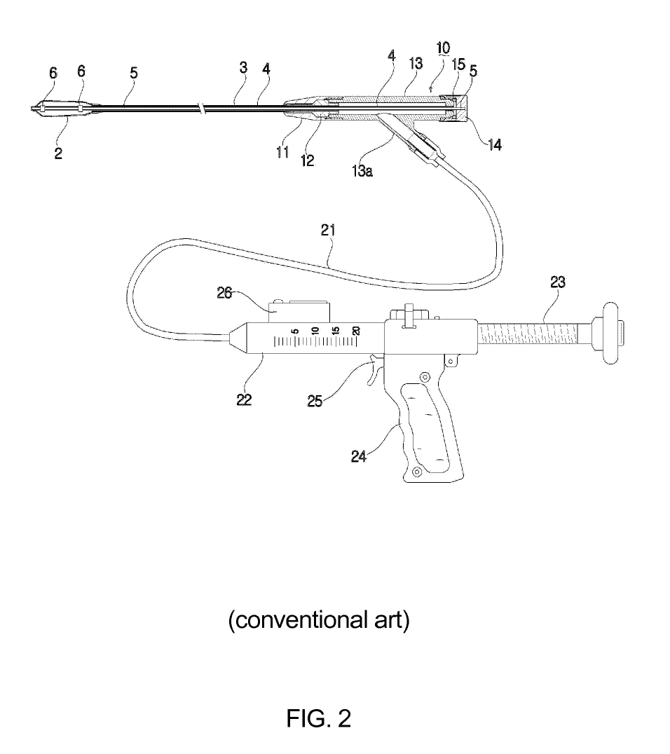

FIG. 3 is a sectional view illustrating a balloon catheter, according to a first embodiment of the present invention;

FIG. 4 is a partially sectional view illustrating a balloon of a balloon catheter, according to a second embodiment of the present invention;

FIG. 5 is a partially sectional view illustrating the balloon catheter and an injector for surgical procedures of the spine which is connected to the balloon catheter according to the first embodiment of the present invention; and

FIG. 6 is of views showing differences in inflation among the balloon of the conventional balloon catheter and the balloons of the balloon catheters of the first and second embodiments.

### DESCRIPTION OF THE PREFERRED EMBODIMENTS

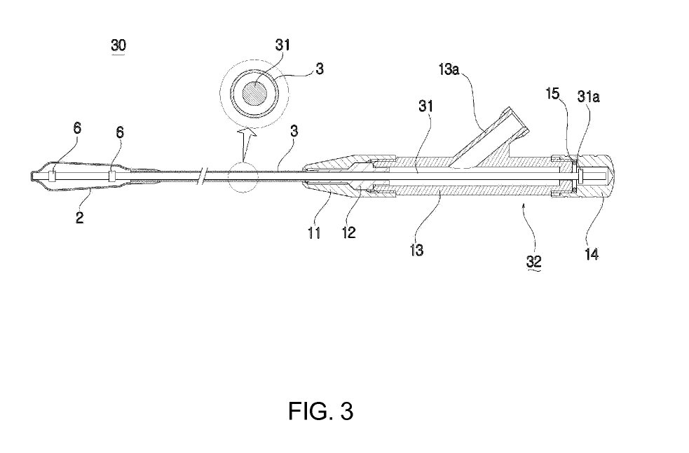

Hereinafter, embodiments of a balloon catheter according to the present invention will be described in detail with reference to FIGS. 3 through 6. Reference should now be made to the drawings, in which the same reference numerals are used throughout the different drawings to designate the same or similar components.

As shown in FIG. 3, a balloon catheter 30 according to an embodiment of the present invention includes an outer tube 3, a rigid rod 31, a balloon 2, markers 6 and a body unit 32.

In detail, the rigid rod 31 is inserted into the outer tube 3 along the length of the outer tube 3 such that a gap is defined between the rigid rod 31 and an inner surface of the outer tube 3. Further, the rigid rod 31 is a solid type rod, front and rear ends of which extend outwards from the outer tube 3 by predetermined distances.

A front end of the balloon 2 is fastened around the front end of the rigid rod 31 and sealed, and a rear end thereof is fastened around a front end of the outer tube 3 and sealed. The front end of the balloon 2 and the front end of the rigid rod 31 or the rear end of the balloon 2 and the front end of the outer tube 3 can be sealed and fastened to each other in such a way that they are fused by heat of a high temperature. Preferably, the balloon 2 is made of thermoplastic polyurethane.

The markers 6 are fastened to the rigid rod 31 in the balloon 2 at opposite positions spaced apart from each other by a predetermined distance. It is preferable for each marker 6 to have a metal ring shape so that it can be easily fitted over and fastened to the rigid rod 31. The markers 6 function to block X-rays so that the location of the balloon 2 can be indicated on the screen of a monitor when irradiating with X-rays.

The body unit 32 is coupled to and sealed to a rear end of the outer tube 3 and supports the rear end of the rigid rod 31 such that there is a gap between the rigid rod 31 and the outer tube 3. The body unit 32 prevents the rigid rod 31 from moving forwards to prevent the balloon 2 from expanding longitudinally during a surgical procedure. Further, in the same manner as described in the background art, the body unit 32 is connected to an injector (not shown) for surgical procedures of the spine and functions to transmit fluid, for example, any one selected from among air, steam, water, etc., from the injector into the balloon 2 through the gap formed between the rigid rod 31 and the outer tube 3.

As such, in place of using the inner tube and the guide wire which are indispensable elements of the conventional art, the balloon catheter 30 according to the present invention is configured such that the solid type rigid rod 31 is provided in the outer tube 3 and the gap is defined between the rigid rod 31 and the outer tube 3, thus simplifying the structure of the balloon catheter 30. Furthermore, in the balloon catheter 30 according to the present invention, because the rigid rod 31 prevents the outer tube 3 from bending, the balloon 2 of the balloon catheter 30 can be easily and precisely positioned in a target area of the spine of a patient without using the inner tube or the guide wire of the conventional art.

Moreover, in the balloon catheter 30 according to the present invention, the rigid rod 31 is of the solid type, so that when fluid of high pressure is used to inflate the balloon 2 during the surgical procedure, the pressure in the balloon 2 is prevented from expanding the rigid rod 31 longitudinally. Hence, the balloon 2 can be appropriately inflated in the radial direction. Here, the term “high pressure of fluid” refers to a pressure capable of inflating the balloon 2 to the appropriate size.

Meanwhile, the balloon catheter according to the present invention may be embodied in a more concrete form by the following embodiments on the basis of the above-mentioned fundamental construction.

It is preferable for the rigid rod 31 to be made of reinforced plastic or fiber-reinforced plastic in consideration of the rigidity, the purchase availability, the weight, the production cost, etc.

As shown in FIG. 3, the body unit 32 includes a cap 11, a stylet 12, a body 13 and a holder 14. The cap 11 is fastened to and sealed to the rear end of the outer tube 3. The stylet 12 is housed in the cap 11 and receives the rigid rod 31 such that the stylet 12 is spaced apart from the outer surface of the rigid rod 31 by a predetermined gap. The body 13 is coupled both to a rear end of the cap 11 and to a rear end of the stylet 12. The holder 14 is coupled to a rear end of the body 13 and sealed thereto by a sealing ring 15 interposed therebetween. The holder 14 retains the rear end of the rigid rod 31 therein. The body 13 has at a predetermined position thereof an inclined port 13a which communicates with the outside. In addition, the body 13 functions to support the rear end of the rigid rod 31.

As shown in FIG. 3, the rear end of the rigid rod 31 that is located in the space between the body 13 and the holder 14 has a stopper 31a thereon or is fastened to the holder 14 to prevent high-pressure fluid which is supplied into the balloon 2 of the balloon catheter 30 from undesirably moving the rigid rod 31 forwards during a surgical procedure. The stopper 31a may be separately manufactured and attached to the rigid rod 31, but it is preferable for the stopper 31a to be integrally formed with the rigid rod 31 for the sake of convenience of manufacture or assembly.

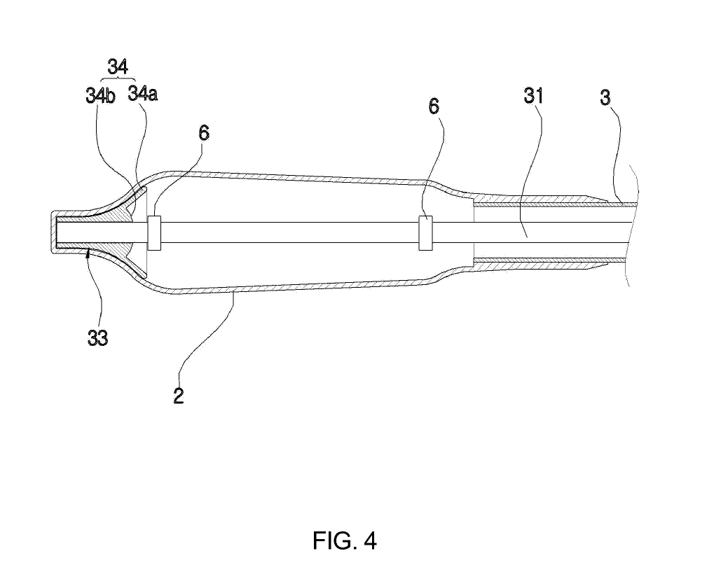

Meanwhile, as shown in FIG. 4, the balloon catheter of the present invention may further include a front tube 33 which is interposed between the inner surface of the front end of the balloon 2 and the front end of the rigid rod 31. The front tube 33 functions to enhance the strength with which the balloon 2 is fixed to the rigid rod 31. Furthermore, the front tube 33 preferably includes on a rear end thereof a pressure diffuser 34 in order to guide high-pressure fluid, which is supplied into the balloon 2 during the surgical procedure, outwards in the radial direction of the balloon 2. The pressure diffuser 34 includes a diffusion guide 34a whose diameter increases from the front end thereof to the rear end, and a curved part 34b which is formed on a central portion of the pressure diffuser 34 and is convex outwards.

Hereinafter, the operation of the balloon catheter according to each embodiment of the present invention will be described with reference to FIGS. 3 through 6.

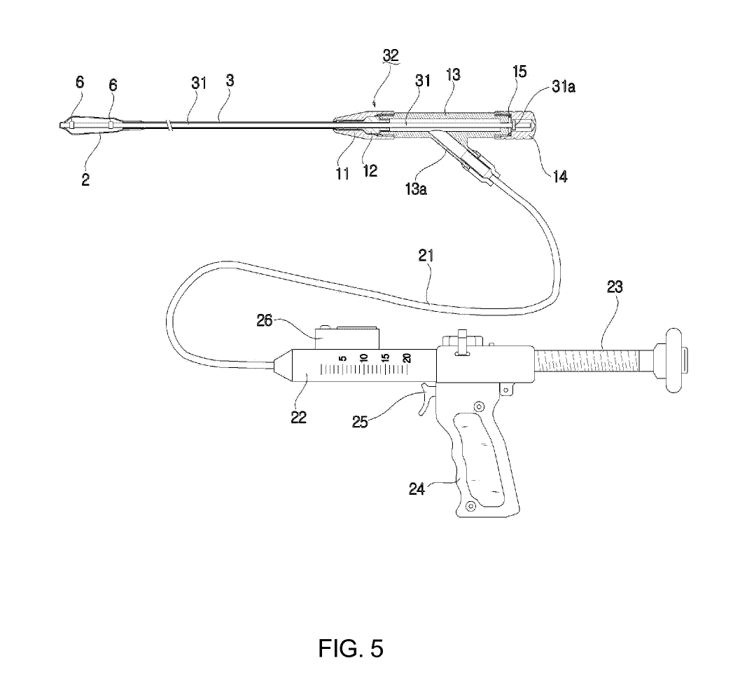

As shown in FIG. 5, after a plunger 23 of an injector 20 for a surgical procedure on the spine has been positioned at a rear end position of a cylinder 22, a connection hose 21 of the injector 20 is connected to the inclined port 13a of the balloon catheter 30 of the present invention, as shown in FIG. 3 or 5.

Subsequently, the catheter 30 is positioned in a target area of the spine of a patient through a catheter passage which has been previously formed in the patient's body by medical instruments. Here, the rigid rod 31 is provided in the outer tube 3 of the catheter 30, which allows the outer tube 3 provided with the balloon 2 on the front end thereof to be smoothly guided in the direction desired by a user rather than bending.

Thereafter, when the user pulls a trigger 25 of the injector 20, the plunger 23 is moved forwards, that is, towards the front end of the cylinder 22. Then, compressed air is supplied from the cylinder 22 to the inclined port 13a of the catheter 30 through the connection hose 21.

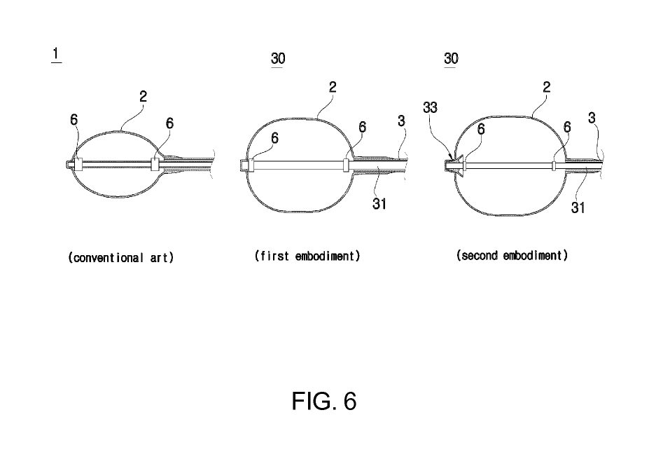

The compressed air that is supplied through the inclined port 13a of the catheter 30 flows along the gap between the outer tube 3 and the rigid rod 31 via the gap between the stylet 12 and the rigid rod 31 and enters the balloon 2 provided on the front end of the outer tube 3, thus inflating the balloon 2 outwards in the radial direction of the balloon 2 (refer to FIG. 6).

In the present invention, the rigid rod 31 is of the solid type, and the front end of the rigid rod 31 is fixed to the balloon 2 while the rear end of the rigid rod 31 is fixed to the holder 14 or held onto the body 13 by the stopper 31a of the rigid rod. Therefore, unlike the balloon catheter 1 according to the conventional art, the balloon 2 can be prevented from inflating in the longitudinal direction of the balloon 2 as high-pressure fluid is injected into the balloon 2.

Meanwhile, as shown in FIG. 4, in the case of the embodiment where the front tube 33 is further provided between the inner surface of the front end of the balloon 2 and the front end of the rigid rod 31, the front tube 33 not only enhances the fixing strength between the balloon 2 and the rigid rod 31 but also appropriately guides high-pressure fluid, supplied into the balloon 2, outwards in the radial direction of the balloon 2, thus helping the balloon 2 inflate radially.

Subsequently, after the balloon 2 has been sufficiently inflated by the above-mentioned process so that an available space is created in the target area of the spine of the patient, an X-ray examination is conducted, for example, to check whether a collapsed bone has returned to its normal location. Thereafter, the balloon 2 of the catheter 30 is extracted from the spine of the patient, and medical cement is injected into the target area of the spine that has recovered its normal form, thus completing the surgical procedure.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

As described above, a balloon catheter according to the present invention is configured in such a way that a rigid rod is disposed in an outer tube, in lieu of using the inner tube and the guide wire which are indispensable elements of the conventional art. Thereby, the structure of the balloon catheter can be markedly simplified compared to the conventional art. Furthermore, the rigid rod which is inserted into the outer tube can facilitate inserting the balloon of the balloon catheter into a target area of the spine of a patient without using the inner tube or the guide wire.

Moreover, because the present invention is provided with the rigid rod inserted into the outer tube, it can prevent an occurrence that happens in the conventional art, namely, the pressure of fluid supplied into the balloon during a surgical procedure inflating the inner tube or the balloon in the longitudinal direction. Thus, in the present invention, the balloon can be appropriately and sufficiently inflated in the radial direction.

In addition, the present invention is further provided with a front tube which is interposed between the inner surface of the front end of the balloon and the front end of the rigid rod. The front tube can further enhance the fixing strength between the front end of the balloon and the rigid rod to which the front end of the balloon is fastened. Also, the front tube can structurally guide fluid injected into the balloon so that the pressure in the balloon can be smoothly applied outwards in the radial direction of the balloon.

What is claimed is:

1. A balloon catheter, comprising:
an outer tube;
a rigid rod having a solid structure and inserted into the outer tube along a length of the outer tube such that a gap is defined between the rigid rod and an inner surface of the outer tube, the rigid rod having front and rear ends extending outwards from the outer tube by predetermined distances;
a balloon fastened at a front end thereof around and sealed to the front end of the rigid rod, the balloon fastened at a rear end thereof around and sealed to a front end of the outer tube;
markers fastened to the rigid rod in the balloon at opposite positions spaced apart from each other by a predetermined distance; and
a body unit fastened to and sealed to a rear end of the outer tube and preventing the rigid rod from moving forwards, the body unit supporting the rear end of the rigid rod such that the gap is defined between the rigid rod and the outer tube so that fluid is supplied from an outside into the balloon through the gap between the outer tube and the rigid rod.

2. The balloon catheter according to claim 1, wherein the rigid rod is made of reinforced plastic or fiber-reinforced plastic.

3. The balloon catheter according to claim 1, wherein the body unit comprises:
a cap fastened to and sealed to the rear end of the outer tube;
a stylet housed in the cap and receiving the rigid rod therein such that a gap is defined between the stylet and an outer surface of the rigid rod;
a body coupled both to a rear end of the cap and to a rear end of the stylet, the body supporting the rear end of the rigid rod, with an inclined port provided at a predetermined position on the body, the inclined portion communicating with the outside; and
a holder coupled to a rear end of the body and sealed to the body by a sealing ring interposed therebetween, the holder retaining the rear end of the rigid body therein.

4. The balloon catheter according to claim 1, further comprising:
a front tube provided between an inner surface of the front end of the balloon and the front end of the rigid rod to enhance a strength with which the balloon and the rigid rod are fixed to each other, the front tube comprising a pressure diffuser on a rear end thereof, the pressure diffuser comprising: a diffusion guide increased in diameter from a front end thereof to a rear end; and a curved part formed on a central portion of the pressure diffuser, the curved part being convex outwards.

5. The balloon catheter according to claim 3, wherein the rear end of the rigid rod that is located in a space between the body and the holder has a stopper thereon or is fastened to the holder, thus preventing high-pressure fluid supplied into the balloon of the balloon catheter from moving the rigid rod forwards during a surgical procedure.
